SEQUENCE LISTING (14 sequences)

SEQ ID NO: 1 - DNA, 28 bp, Artificial Sequence
  Primer for control
aacccacttcttccacagtttcttctaa

SEQ ID NO: 2 - DNA, 22 bp, Artificial Sequence
  Primer for control
tggcatcgcatagcattctttg

SEQ ID NO: 3 - DNA, 26 bp, Artificial Sequence
  Primer for control
tctttttaaacctccggaacccactt

SEQ ID NO: 4 - DNA, 25 bp, Artificial Sequence
  Primer for control
ggatggcatcgcatagcattctttg

SEQ ID NO: 5 - DNA, 21 bp, Neisseria gonorrhoeae
gatccaactattcccgattgc

SEQ ID NO: 6 - DNA, 24 bp, Neisseria gonorrhoeae
gcaaagttatacagcttcgcctga

SEQ ID NO: 7 - DNA, 19 bp, Neisseria gonorrhoeae
caactattcccgattgcga

SEQ ID NO: 8 - DNA, 20 bp, Neisseria gonorrhoeae
gttatacagcttcgcctgaa

SEQ ID NO: 9 - DNA, 17 bp, Chlamydia trachomatis
cgtaccagaaggagcag

SEQ ID NO: 10 - DNA, 18 bp, Chlamydia trachomatis
cgtctccaggacaacgtc

SEQ ID NO: 11 - DNA, 25 bp, Chlamydia trachomatis
tgctctcagagtttggacagttcct

SEQ ID NO: 12 - DNA, 20 bp, Chlamydia trachomatis
tttcttggcgggtgcagaca

SEQ ID NO: 13 - DNA, 368 bp, Neisseria gonorrhoeae
tctttttaaacctccggaacccacttcttccacagattcttctaaagaacctcctaaagaatctgcatggaaagtagtctctcattctcgaggacgccgtcgcgctcgatccaacccctcccctcacacatctcaaaatactccttctccaaaagactcttctttagttgctcgtacggataaagcggcaacagatatctttaattcggctaaacacaaagcgattgaaacgacaaaaagaagtgatcagcaaagcagatccttacatatactgcaccttttagctgaaaatccggaacccattgtgttccactcagctcaccaaacaaaccacaacgatccgcaaagaatgctatgcgatgccatcc

SEQ ID NO: 14 - DNA, 261 bp, Chlamydia trachomatis
gcaactattcccgattgcgacatcattttaggcggattcccttgtcaagatttttccatgatttggaaacagccgggcttagagggtgagcgcggcaatctttataaaagctttttacgttttgtaaatgcaaaaaaaccgaaagtttttgttgctgagaatgtgaaaggtttattgactgccaacaagaaaaaagccatccagcaaattattaccgactttgaaaattgcggttattacgttcaggcgaagctgtataac

(19) United States
(12) Patent Application Publication
(10) Pub. No.: US 2010/0311049 A1
(43) Pub. Date: Dec. 9, 2010

(54) PCR-Based Kit For Detecting Chlamydia Trachomatis and Nelsseria Gonorrhoeae

(75) Inventors: Daman Saluja, New Delhi (IN); Uma Chaudhary, New Delhi (IN); Mashook Ali, New Delhi (IN); Poonam Sachdeva, New Delhi (IN); Achchhe Lal Patel, New Delhi (IN)

Correspondence Address: THE WEBB LAW FIRM, P.C., 700 KOPPERS BUILDING, 436 SEVENTH AVENUE, PITTSBURGH, PA, 15219, US

(73) Assignees: UNIVERSITY OF DELHI, NEW DELHI (IN); DEPARTMENT OF BIOTECHNOLOGY, NEW DELHI (IN)

(21) Appl. No.: 12/651,819

(22) Filed: Jan. 4, 2010

Related U.S. Application Data
(63) Continuation-in-part of application No. 11/436,063, filed on May 17, 2006, now abandoned.

(30) Foreign Application Priority Data
Dec. 22, 2005 (IN) 3437/DEL/05

(51) Int. Cl. C12Q 1/68

(52) U.S. Cl. 435/6

(57) ABSTRACT

The invention relates to a PCR based prototype kit for detecting Chlamydia trachomatis and Neisseria gonorrhoeae comprising: a transport medium for sample collection solution A and B, a reaction mixture having the primer for Chlamydia trachomatis and Neisseria gonorrhoeae, a gel loading dye, Agarose gel, gel running buffer and a DNA marker ladder.

### FIELD OF INVENTION

This invention relates to a PCR-based prototype kit for detecting chlamydia trachomatis and Neisseria gonorrhoeae.

### BACKGROUND OF THE INVENTION

In India Chlamydia trachomatis and Neisseria gonorrhoeae, are detected by separate method in spite of the fact about 50% of the sample are co-infected. The usual method of detection is Gram-staining followed by confirmation like, antigen detection or biochemical assay. Both these methods are highly unsatisfactory, especially in asymptomatic patient (where the infection load is low). At present in India few private pathological laboratory are carrying out PCR based diagnostic, for which they are completely dependent on the import of kit. There is no indigenous diagnostic kit available for Chlamydia trachomatis and Neisseria gonorrhoeae at present.

The drawbacks of the term existing state of art is as follows:

Culture method: Sensitivity of this method is as low as 50% as organisms may lose infectivity during transportation and storage, which will reduce the likelihood of propagation. In addition, the surface area of the cell culture layer and/or the amount of sample material added to the cell culture influence the sensitivity. Cell culture, however, is time-consuming, laborious and expensive and can therefore be provided by only a few central laboratories.

Antigen Detection: The diagnostic efficacy of these methods is not high enough to warrant clinical use unless the need for a fast result overweighs the lower diagnostic accuracy. Also, the ELISA tests may reveal positive results in the presence of other organisms such as E. coli and Bacteroides sp, and Staphylococcus aureus may be captured instead of Chlamydia due to binding to the Fc region of the antibodies, thereby causing false-positive reactions. DFA requires skilled personnel in order to differentiate C. trachomatis organisms from non-specific fluorescent particles.

DNA/RNA Detection: The diagnostic performance of non-amplified probe technique is not substantially different from that of the best ELISA.

Nucleic acid amplification tests (NAATs): Target gene for NAATs The Plasmid: Some studies give evidence or suggest that the plasmid-free variants are present in clinical samples, and although it may seem that plasmid is involved in DNA replication, it has been possible to culture a plasmid-free variant. Thus, the infections caused by plasmid-free variants will be undetected if the plasmid is used as target gene.

The 16S-rRNA gene: Due to high homology of the 16S rRNA gene with other organisms, optimal reaction conditions are crucial in order to avoid annealing of primers to 16S-rRNA genes of the other organisms that are present in all non-sterile clinical samples.

### OBJECTS OF THE INVENTION

An object of this invention is to propose a PCR-based prototype kit for detecting Chlamydia trachomatis and Neisseria gonorrhoeae simultaneously and individually.

Another object of this invention is to propose a kit which is cost effective.

Further object of this invention is to propose a kit which reduces the chances of error and any cross contamination.

Still further object of this invention is to propose a kit which can be operated without any technical expertise.

### SUMMARY OF THE INVENTION

According to this invention there is provided a PCR based prototype kit for detecting Chlamydia trachomatis and Neisseria gonorrhoeae comprising;

A transport medium for sample collection solution A & B, a reaction mixture having the primers for Chlamydia trachomatis & Neisseria gonorrhoeae, a gel loading dye, Agarose gel, gel running buffer and a DNA marker ladder.

### DETAILED DESCRIPTION OF THE INVENTION

In the present invention a multiplex PCR based prototype kit for the detection of the Chlamydia trachomatis and Neisseria gonorrhoeae, based on designed primers (patent in process). The kit contains all the reagents for collection of clinical samples, for carrying out amplification by PCR and the detection of the product, as well as the protocol to be used for diagnosis of Chlamydia trachomatis and Neisseria gonorrhoeae in patient samples. Furthermore, three kits have been developed:

Kit for diagnosis of samples infected with Chlamydia trachomatis with an internal control.

Kit for diagnosis of sample infected with Chlamydia trachomatis without internal control.

Kit for detection of Chlamydia trachomatis and Neisseria gonorrhoeae simultaneously.

In one embodiment, the invention is directed to primers for diagnosing of sample infected with Chlamydia trachomatis with an internal control comprising primers SEQ ID No.1 and 2 or 3 and 4 with 9 and 10 or 11 and 12 and Primers for diagnosis of sample infected with Chlamydia trachomatis without internal control comprising primers SEQ ID No.1 and 2 or 3 and 4.

In another embodiment, the invention is directed to primers for detection of Chlamydia trachomatis and Neisseria gonorrhoeae simultaneously with internal control comprising primers SEQ ID No.1 and 2 or 3 and 4 for Chlamydia trachomatis, SEQ ID No.5 and 6 or 7 and 8 for Neisseria gonorrhoeae, with 9 and 10 or 11 and 12.

In principle any pair of oligonucleotide which bind to the two strand of duplex DNA in sufficiently close proximity could be used as primers for PCR but in practice no guarantee of success in selecting suitable primer set for PCR. However, it is known that many theoretically suitable primer sets simply do not work in practice for co-amplification.

The primer sets identified have established that they are specific and complimentary to the target nucleic acid to form a desired hybridization products and then be extendable by DNA polymerase in practice under routine use.

However, inventors are using exactly complimentary primer sets to the target site of genomic DNA sequences of Neisseria gonorrhoeae and Chlamydia trachomatis for designing a diagnostic method using crude samples while cryptic plasmid is being used in most of the existing methods.

So the novelty of the method is the identification of unique, specific, sensitive yet cost effective PCR based detection of Gonococcal and Chlamydial infection.

The indicated length of orf1 and phospholipase D endonuclease is the size of amplicon obtained from said genes for the detection of Neisseria gonorrhoeae and Chlamydia trachomatis. The Primer region of genes have been chosen in non variable which is present in most of the species of Neisseria gonorrhoea and Chlamydia trachomatis infecting human.

The 10% of positive samples were confirmed by sequencing and sequences were showed 100% homology to existing sequence of orf1 gene of Neisseria gonorrhoeae and phospholopase D endonuclease gene of Chlamydia trachomatis.

Primers sequences of Phospholipase D Endonuclease Superfamily gene, used in diagnosis of Chlamydia trachomatis generates 368-bp product.

Preferred primers

| | |
|---|---|
| 5′-AACCCACTTCTTCCACAGTTTCTTCTAA-3′ | (SEQ ID NO: 1) |
| 5′-TGGCATCGCATAGCATTCTTTG-3′ | (SEQ ID NO: 2 |
| Alternate primers | |
| 5′-TCTTTTTAAACCTCCGGAACCCACTT-3′ | (SEQ ID NO: 3) |
| 5′-GGATGGCATCGCATAGCATTCTTTG-3′ | (SEQ ID NO: 4) |

Amplified region of gene used in diagnosis

(SEQ ID NO: 13)
TCTTTTTAAACCTCCGGAACCCACTTCTTCCACAGATTCTTCTAAAGAAC
CTCCTAAAGAATCTGCATGGAAAGTAGTCTCTCATTCTCGAGGACGCCGT
CGCGCTCGATCCAACCCCTCCCCTCACACATCTCAAAATACTCCTTCTCC
AAAAGACTCTTCTTTAGTTGCTCGTACGGATAAAGCGGCAACAGATATCT
TTAATTCGGCTAAACACAAAGCGATTGAAACGACAAAAAGAAGTGATCAG
CAAAGCAGATCCTTACATATACTGCACCTTTTAGCTGAAAATCCGGAACC
CATTGTGTTCCACTCAGCTCACCAAACAAACCACAACGATCCGCAAAGAA
TGCTATGCGATGCCATCC.

Primers sequences of Orfl gene used in diagnosis of Neisseria gonorrhoeae, generates 250-by product.

| | |
|---|---|
| Preferred primers | |
| 5′-GATCCAACTATTCCCGATTGC-3′ | (SEQ ID NO: 5) |
| 5′-GCAAAGTTATACAGCTTCGCCTGA-3′ | (SEQ ID NO: 6) |
| Alternate primers | |
| 5′-CAACTATTCCCGATTGCGA-3′ | (SEQ ID NO: 7) |
| 5′-GTTATACAGCTTCGCCTGAA-3′ | (SEQ ID NO: 8) |

Amplified region of gene used in diagnosis

(SEQ ID NO: 14)
GCAACTATTCCCGATTGCGACATCATTTTAGGCGGATTCCCTTGTCAAGA
TTTTTCCATGATTTGGAAACAGCCGGGCTTAGAGGGTGAGCGCGGCAATC
TTTATAAAAGCTTTTTACGTTTTGTAAATGCAAAAAAACCGAAAGTTTTT
GTTGCTGAGAATGTGAAAGGTTTATTGACTGCCAACAAGAAAAAAGCCAT
CCAGCAAATTATTACCGACTTTGAAAATTGCGGTTATTACGTTCAGGCGA
AGCTGTATAAC.

The amplified region is 368 bp. When amplified in a sample, the sample is positive for Chlamydia trachomatis

Internal Control

Primers sequences used in prototype kit as a internal control.

| | |
|---|---|
| Alternate sequences | |
| 5′-CGTACCAGAAGGAGCAG-3′ | (SEQ ID NO: 9) |
| 5′-CGTCTCCAGGACAACGTC-3′ | (SEQ ID No: 10) |
| Preferred sequences | |
| 5′-TGCTCTCAGAGTTTGGACAGTTCCT-3′ | (SEQ ID No: 11) |
| 5′-TTTCTTGGCGGGTGCAGACA-3′ | (SEQ ID NO: 12) |

General information:

I. Information for SEQ ID NO: 1:

Sequence Characteristics

(A) LENGTH: 28 base pairs

(B) TYPE: nucleic acid

(C) STRANDEDNESS: single

(D) TOPOLOGY: linear

(E) SEQUENCE DESCRIPTION: SEQ ID NO: 1: 5′-AACCCACTTCTTCCACAGTTTCTTCT AA-3′

2. Information for SEQ ID NO: 2:

Sequence Characteristics

(A) LENGTH: 22 base pairs

(B) TYPE: nucleic acid

(C) STRANDEDNESS: single

(D) TOPOLOGY: linear

(E) SEQUENCE DESCRIPTION: SEQ ID NO: 2: 5′-TGGCATCGCATAGCATTCTTTG-3′

3. Information for SEQ ID NO:3:

Sequence Characteristics

(A) LENGTH: 26 base pairs

(B) TYPE: nucleic acid

(C) STRANDEDNESS: single

(D) TOPOLOGY: linear

(E) SEQUENCE DESCRIPTION: SEQ ID NO:3: 5′-TCTTTTTAAACCTCCGGAACCCACTT-3′

4. Information for SEQ ID NO: 4:

Sequence Characteristics

(A) LENGTH: 25 base pairs

(B) TYPE: nucleic acid

(C) STRANDEDNESS: single

(D) TOPOLOGY: linear

(E) SEQUENCE DESCRIPTION: SEQ ID NO:4: 5′-GGATGGCATCGCATAGCATTCTTTG-3′

5. Information for SEQ ID NO: 5:

Sequence Characteristics

(A) LENGTH: 21 base pairs

(B) TYPE: nucleic acid

(C) STRANDEDNESS: single

(D) TOPOLOGY: linear

(E) SEQUENCE DESCRIPTION: SEQ ID NO:5: 5′ GA TCCAACTATTCCCGATTGC-3′

6. Information for SEQ ID NO: 6:

Sequence Characteristics

(A) LENGTH: 24 base pairs

(B) TYPE: nucleic acid

(C) STRANDEDNESS: single

(0) TOPOLOGY: linear

(E) SEQUENCE DESCRIPTION: SEQ ID NO:6: 5′-GCAAAGTT ATACAGCTTCGCCTGA-3′

7. Information for SEQ ID NO: 7:

Sequence Characteristics

(A) LENGTH: 20 base pairs

(B) TYPE: nucleic acid

(C) STRANDEDNESS: single

(D) TOPOLOGY: linear

(E) SEQUENCE DESCRIPTION:SEQ ID NO:7: 5′-CCTGATGCTAGGGACGGATT-3′

8. Information for SEQ ID NO: 8:

Sequence Characteristics

(A) LENGTH: 22 base pairs

(B) TYPE: nucleic acid

(C) STRANDEDNESS: single

(D) TOPOLOGY: linear

(E) SEQUENCE DESCRIPTION: SEQ ID NO:8: 5′-CCCTAAATTATGCGGTGGAAT-3′

9. Information for SEQ ID NO: 9:

Sequence Characteristics

(A) LENGTH: 17 base pairs

(B) TYPE: nucleic acid

(C) STRANDEDNESS: single

(D) TOPOLOGY: linear

(E) SEQUENCE DESCRIPTION:SEQ ID NO:9: 5′-CGTACCAGAAGGAGCAG-3′

10. Information for SEQ ID NO: 10:

Sequence Characteristics

(A) LENGTH: 18 base pairs

(B) TYPE: nucleic acid

(C) STRANDEDNESS: single

(D) TOPOLOGY: linear

(E) SEQUENCE DESCRIPTION:SEQ ID NO:10: 5′-CGTCTCCAGGACAACGTC-3′

11. Information for SEQ ID NO:11:

Sequence Characteristics

(A) LENGTH: 17 base pairs

(B) TYPE: nucleic acid

(C) STRANDEDNESS: single

(D) TOPOLOGY: linear

(E) SEQUENCE DESCRIPTION: SEQ ID NO:11: 5′-TGCTCTCAGAGTTTGGACAGTTCCT-3′

12. Information for SEQ ID NO:12:

Sequence Characteristics

(A) LENGTH: 20 base pairs

(B) TYPE: nucleic acid

(C) STRANDEDNESS: single

(D) TOPOLOGY: linear

(E) SEQUENCE DESCRIPTION: SEQ ID NO:12: 5′-TTTCTTGGCGGGTGCAGACA-3′

This present invention has been explained in greater detail in the examples:

### Example 1

### Prototype Kit One for Diagnosis of Chlamydia trachomatis

Kit with internal control.

Protocol 1

Sample Collection:

Sample is collected as a swab and is dipped in the vial containing sterile 1 ml Transport medium and stored at 4° C. for several hours or freezer for a maximum time up to one week.

Preparation of Sample DNA:
1. Take 500 μl of collected sample cocktail, in a fresh microcentrifuge tube (1.5 ml) and centrifuge at 15,000 g, for 30 minute at 4° C.
2. Discard the supernatant carefully without disturbing the pellet.
3. Mixed the appropriate amount of solution (Solution A, 48 μl+Solution B, 2 μl) before use.
4. Suspend the pellet from step 3 in 50 μl of the above solution.
5. Incubated at 37° C. for 1 hr.
6. Now incubate the sample at 100° C. for 10 minute.
7. Spin at 10,000 rpm for 2 minute.
8. Collect the supernatant carefully in separate tube (Designated as Sample DNA).

Setting up the PCR:

| | |
|---|---|
| Sample DNA | 08 μl |
| Reaction mixture I | 26 μl |
| Reaction mixture II | 16 μl |

PCR program:

| | Step | Temp | Duration |
|---|---|---|---|
| One cycle | 1 | 94° C. | 5 minutes |
| 35 cycles of | 2 | 95° C. | 30 seconds |
| step 2 to 4, | 3 | 63° C. | 30 seconds |
| | 4 | 72° C. | 30 seconds |
| One cycle | 5 | 72° C. | 5 minute |
| | 6 | 4° C. | Tubes can be taken out after |
| | | | keeping for 10 minute at 4° C. |

After completing the PCR reaction, add 10 μl of the loading dye. Fractionate the product on the agarose gel (1.5%), by loading 25-30 μl the sample per well as described below.

Preparation of Agarose Gel:

Protocol:
1. To prepare 100 ml of a 1.5% agarose solution, weigh 1.5 g agarose into a glass beaker or flask and add 100 ml 1× Running buffer (Dilute from 50× Running buffer before use
2. Microwave or stir on a hot plate until agarose is dissolved and solution is clear.
3. Allow solution to cool to about 55° C. before pouring.
4. Prepare gel tray by sealing ends with tape or other custom-made dam.
5. Place comb in gel tray about 1 inch from one end of the tray and position the comb vertically such that the teeth are about 1-2 mm above the surface of the tray.
6. Pour gel solution into tray to a depth of about 5 mm. Allow gel to solidify about 20 minutes at room temperature.
7. To run, gently remove the comb, place tray in electrophoresis chamber, and cover (just until wells are submerged) with 1× Running buffer (the same buffer used to prepare the agarose)
8. Load 25-30 μl of PCR product with loading dye per well. Use one lane for loading 5 μl of the marker DNA.
9. Electrophoreses at 50-150 volts until orange dye have migrated an appropriate distance (about 5-6 cms).
10. Excess agarose can be stored at room temperature and remelted in a microwave and can be used again.

Result Analysis:
A. All reaction, give PCR product of 800 base pair for internal control. This proves PCR reaction is working fine and there is no inhibitor of PCR in the clinical sample.
B. Only Chlamydia trachomatis positive sample, give an extra band of 368 bp as is evident from the DNA size marker lane.

Alternate protocol for sample preparation from clinical samples.

Protocol 2

Preparation of Sample DNA:
1. Take 500 μl of collected sample cocktail, in a fresh microcentrifuge tube (1.5 ml) and centrifuge at 15,000 g, for 30 minute at 4° C.
2. Discard the supernatant carefully without disturbing the pellet.
3. Suspend the pellet from step 2 in 50 μl of TE solution.
4. Now incubate the sample at 100° C. for 10 minute
5. Spin at 10,000 rpm for 2 minute.
6. Collect the supernatant carefully in separate tube (Designated as Sample DNA).

All other steps will be as mentioned in the detailed assay.

Components Supplied in the Kit One:
1. Transport Medium for sample collection
2. Solution A
3. Solution B
4. Reaction mixture I
5. Reaction mixture II
6. Gel loading dye
7. Agarose gel
8. Gel running buffer (50×)
9. DNA marker ladder
10. TE solution
11. Protocol 1
12. Protocol 2

Annexure:2

Prototype kit two for diagnosis of Chlamydia Trachomatis

Kit II Without Internal Control

Protocol 1

Sample Collection:

Sample is collected as a swab and is dipped in the vial containing sterile 1 ml Transport medium and stored at 4° C. for several hours or in freezer for a (maximum time up to one week).

Preparation of Sample DNA:
1. Take 500 μl of collected sample cocktail, in a fresh microcentrifuge tube (1.5 ml) and centrifuge at 15,000 g, for 30 minute at 4° C.
2. Discard the supernatant carefully without disturbing the pellet.
3. Mixed the appropriate amount of solution (Solution A, 48 μl Solution B, 2 μl) before use.
4. Suspend the pellet from step 3 in 50 μl of the above solution.
5. Incubated at 37° C. for 1 hr.
6. Now incubate the sample at 100° C. for 10 minute
7. Spin at 10,000 rpm for 2 minute.
8. Collect the supernatant carefully in separate tube (Designated as Sample DNA).

Setting up the PCR:

| | |
|---|---|
| Sample DNA | 08 μl |
| Reaction mixture I | 26 μl |
| Reaction mixture II | 16 μl |

PCR program:

| | Step | Temp | Duration |
|---|---|---|---|
| One cycle | 1 | 94° C. | 5 minutes |
| 35 cycles of | 2 | 95° C. | 30 seconds |
| step 2 to 4, | 3 | 63° C. | 30 seconds |
| | 4 | 72° C. | 30 seconds |
| One cycle | 5 | 72° C. | 5 minutes |
| | 6 | 4° C. | Tubes can be taken out |
| | | | after keeping for |
| | | | 10 minute at 4° C. |

After completing the PCR reaction, add 10 μl of the loading dye. Fractionate the product on the agarose gel (1.5%), by loading 25-30 μl the sample per well as described below.

Protocol:
1. To prepare 100 ml of a 1.5-% agarose solution, weigh 1.5-g agarose into a glass beaker or flask and add 100 ml 1× Running buffer. (Dilute from 50× gel running buffer before use)
2. Microwave or stir on a hot plate until agarose is dissolved and solution is clear.
3. Allow solution to cool to about 55° C. before pouring.
4. Prepare gel tray by sealing ends with tape or other custom-made dam.
5. Place comb in gel tray about 1 inch from one end of the tray and position the comb vertically such that the teeth are about 1-2 mm above the surface of the tray
6. Pour gel solution into tray to a depth of about 5 mm. Allow gel to solidify about 20 minutes at room temperature.
7. To run, gently remove the comb, place tray in electrophoresis chamber, and cover (just until wells are submerged) with 1× Running buffer (the same buffer used to prepare the agarose)
8. Load 25-30 μl of PCR product with loading dye per well. Use one lane for loading 5 μl of the marker DNA.
9. Electrophoreses at 50-150 volts until orange dye have migrated an appropriate distance (about 5-6 cms).
10. Excess agarose can be stored at room temperature and remelted in a microwave and can be used again.

Result Analysis:

Only Chlamydia trachomatis positive sample give a band of 368 by as is evident from the DNA size marker lane.

Components Supplied in the Kit:
1. Transport medium for sample collection
2. Solution A
3. Solution B
4. Reaction mixture I
5. Reaction mixture II
6. Gel loading dye
7. Agarose gel
8. Gel running buffer (50×)
9. DNA marker ladder
10. TE solution
11. Protocol 1
12. Protocol 2

Alternate protocol for sample preparation from clinical samples.

Protocol 2

Preparation of Sample DNA:
1. Take 500 μl of collected sample cocktail, in a fresh microcentrifuge tube (1.5 ml) and centrifuge at 15,000 g, for 30 minute at 4° C.
2. Discard the supernatant carefully without disturbing the pellet.
3. Suspend the pellet from step 2 in 50 μl of TE solution.
4. Now incubate the sample at 100° C. for 10 minute
5. Spin at 10,000 rpm for 2 minute.
6. Collect the supernatant carefully in separate tube (Designated as Sample DNA).

All other protocols will be as mentioned in the detailed assay.

Annexure: 3

Prototype Kit III

For diagnosis of Chlamydia Trachomatis and Neisseria Gonorrhoeae Kit III with internal control.

Protocol

Sample Collection:

Sample is collected as a swab and is dipped in the vial containing sterile 1 ml Transport medium and stored at 4° C. for several hours or in freezer for a (maximum time up to one week).

Preparation of Sample DNA:
1. Take 500 μl of collected sample cocktail, in a fresh microcentrifuge tube (1.5 ml) and centrifuge at 15,000 g, for 30 minute at 4° C.
2. Discard the supernatant carefully without disturbing the pellet.
3. Mixed the appropriate amount of solution (Solution A, 48 μl+ Solution B, 2 μl) before use.
4. Suspend the pellet from step 3 in 50 μl of the above solution.
5. Incubated at 37° C. for 1 hr.
6. Now incubate the sample at 100° C. for 10 minute.
7. Spin at 10,000 rpm for 2 minute.
8. Collect the supernatant carefully in separate tube (Designated as Sample DNA).

Setting up the PCR:

| | |
|---|---|
| Sample DNA | 08 μl |
| Reaction mixture 1 | 26 μl |
| Reaction mixture II | 16 μl |

PCR program:

| | Step | Temp | Duration |
|---|---|---|---|
| One cycle | 1 | 94° C. | 5 minutes |
| 35 cycles of | 2 | 94° C. | 45 seconds |
| step 2 to 4, | 3 | 50° C. | 45 seconds |
| | 4 | 72° C. | 45 seconds |
| One cycle | 5 | 72° C. | 5 minutes |
| | 6 | 4° C. | Tubes can be taken out |
| | | | after keeping for 10 |
| | | | minutes at 4° C. |

After completing the PCR reaction, add 10 μl of the loading dye. Fractionate the product on the agarose gel (1.5%), by loading 25-30 μl the sample per well as described below.

Protocol:
1. To prepare 100 ml of a 1.5-% agarose solution, weigh 1.5-g agarose into a glass beaker or flask and add 100 ml 1× Running buffer (Dilute from 50× running buffer by taking 98 ml water and 2 ml of 50× running buffer).
2. Microwave or stir on a hot plate until agarose is dissolved and solution is clear.
3. Allow solution to cool to about 55° C. before pouring.
4. Prepare gel tray by sealing ends with tape or other custom-made dam.
5. Place comb in gel tray about 1 inch from one end of the tray and position the comb vertically such that the teeth are about 1-2 mm above the surface of the tray.
6. Pour gel solution into tray to a depth of about 5 mm. Allow gel to solidify about 20 minutes at room temperature.
7. To run, gently remove the comb, place tray in electrophoresis chamber, and cover (just until wells are submerged) with 1× Running buffer (the same buffer used to prepare the agarose)
8. Load 25-30 μl of PCR product with loading dye per well. Use one lane for loading 5 μl of the marker DNA.
9. Electrophoreses at 50-150 volts until orange dye have migrated an appropriate distance (about 5-6 cms).
10. Excess agarose can be stored at room temperature and remelted in a microwave and can be use again.

Result Analysis:
A. All reaction, give PCR product of 800 base pair for internal control. This proves PCR reaction is working fine and there is no inhibitor of PCR in the clinical sample.
B. Chlamydia trachomatis, Neisseria gonorrhoeae positive sample, gives two extra band of 368 bp for Chlamydia trachomatis and 260 bp for Neisseria gonorrhoeae, as is evident from the DNA size marker lane.
C. Only Chlamydia trachomatis, positive sample, gives one extra band of 368 bp. as is evident from the DNA size marker lane.
D. Only Neisseria gonorrhoeae, positive sample, gives one extra band of 260 bp, as is evident from the DNA size marker lane.

Components Supplied in the Kit:
1. Transport medium for sample collection
2. Solution A
3. Solution B
4. Reaction mixture I
5. Reaction mixture II
6. Gel loading dye
7. Agarose gel
8. Gel running buffer 950×)
9. DNA marker ladder
10. Protocol

We claim:

1.-8. (canceled)

9. A PCR based prototype kit for detecting Chlamydia trachomatis and Neisseria gonorrhoeae comprising:
a transport medium for sample collection solution A and B, a reaction mixture comprising a primer for Chlamydia trachomatis and Neisseria gonorrhoeae, a gel loading dye, Agarose gel, gel running buffer and a DNA marker ladder, wherein the primer is an isolated nucleic acid having a sequence selected from the group consisting of SEQ ID NOs: 1-12.

10. The PCR based prototype kit as claimed in claim 9, wherein a positive sample of Chlamydia trachomatis gives a band of 368 by as is shown by a DNA marker ladder.

11. The PCR based prototype kit as claimed in claim 9, wherein a positive sample of Neisseria gonorrhoeae gives a band of 260 bp.

12. The PCR based prototype kit as claimed in claim 9, wherein said solution A is Tris (50 mm), EDTA(IMM) and Tritonx100 (1%); and solution B is proteinase k(200 μg/ml).

13. The PCR based prototype kit as claimed in claim 9, wherein the gel loading dye comprises:

| | |
|---|---|
| Tris HC1 | 120 mM |
| Orange G | 1.5% |
| Xylene Cynol FF | 0.03% |
| Glycerol | 60% |
| EDTA | 60 mM |

and the gel running buffer is Trisbase (242 g), EDTA (50 mM, pH8.0) and glacialaciticacid (100 ml).

14. The PCR based prototype kit as claimed in claim 9, wherein the reaction mixture further comprises a second primer have a sequence selected from the group consisting of SEQ ID NOs: 1-4.

15. The PCR based prototype kit as claimed in claim 14, wherein the reaction mixture further comprises a third primer have a sequence selected from the group consisting of SEQ ID NOs: 5-8.

16. A method for detecting Chlamydia trachomatis and Neisseria gonorrhoeae in a sample comprising:
collecting the sample as a swab in a transport medium;
mixing said sample with solution A and solution B;
subjecting said mixture to the step of incubation;
preparing a sample DNA;
treating the sample DNA with the reaction mixture comprising detecting Chlamydia trachomatis and Neisseria gonorrhoeae in the sample, wherein the reaction mixture comprises a primer having a sequence selected from the group consisting of SEQ ID NOs: 1-12.

17. The method as claimed in claim 14, wherein the amount of said solution A is 48 μl and the amount of solution B is 2 μl.

18. The method as claimed in claim 14, wherein the step of incubation is at 100° C. for 10 minutes.

19. The method as claimed in claim 14, wherein the reaction mixture further comprises a second primer have a sequence selected from the group consisting of SEQ ID NOs: 1-4.

20. The method as claimed in claim 14, wherein the reaction mixture further comprises a third primer have a sequence selected from the group consisting of SEQ ID NOs: 5-8.

21. A composition for diagnosing a sample infected with Chlamydia trachomatis comprising a first pair of primers having sequences of SEQ ID NOs.: 1 and 2, 3 and 4, 9 and 10, or 11 and 12.

22. The composition as claimed in claim 21 further comprising a second pair of primers having sequences of SEQ ID NOs: 1 and 2, 3 and 4, 9 and 10, or 11 and 12.

23. A composition for detecting Chlamydia trachomatis and Neisseria gonorrhoeae simultaneously comprising a first set of primers having sequences of: SEQ ID NOs.: 1 and 2, or 3 and 4 for Chlamydia trachomatis; a second set of primers having sequences of SEQ ID NOs.: 5 and 6, or 7 and 8 for Neisseria gonorrhoeae.

24. The composition as claimed in claim 23 further comprising a third set of primers having sequences of SEQ ID NOs.: 9 and 10, or 11 and 12.
